(19) United States
(12) Patent Application Publication
(10) Pub. No.: US 2014/0256754 A1
(43) Pub. Date: Sep. 11, 2014

(54) QUINAZOLINES AS B-GLUCURONIDASE NOVEL INHIBITORS

(71) Applicants: Muhammad Iqbal Choudhary, Karachi (PK); Kahalid Mohammed KHAN, Karachi (PK); Nimra Naveed SHAIKH, Karachi (PK); Syed Muhammad SAAD, Karachi (PK); Sammer YOUSUF, Karachi (PK); Atta-ur Rahman, Karachi (PK)

(72) Inventors: Muhammad Iqbal Choudhary, Karachi (PK); Kahalid Mohammed KHAN, Karachi (PK); Nimra Naveed SHAIKH, Karachi (PK); Syed Muhammad SAAD, Karachi (PK); Sammer YOUSUF, Karachi (PK); Atta-ur Rahman, Karachi (PK)

(21) Appl. No.: 13/789,021

(22) Filed: Mar. 7, 2013

(51) Int. Cl. C07D 239/91

(52) U.S. Cl. CPC C07D 239/91
USPC 514/266.31; 435/184

(57) ABSTRACT

Quinazoline derivatives 1-25, (2-[3,4-bis(methyloxy)phenyl]quinazolin-4-(3H)-one) and 2-[2-(ethyloxy)phenyl]quinazoline-4-(3H)-one) are reported as β-glucuronidase inhibitors useful in the treatment of β-glucuronidase hyperactivity disorders.

### FIELD OF THE INVENTION

The present invention relates to quinazolines class of compounds and its derivatives as novel β-glucuronidase inhibitors in order to treat various health disorders related to over expression of β-glucuronidase enzyme.

### BACKGROUND OF THE INVENTION

Glucuronidation is known as a defensive process of the body to excrete the toxic chemicals from the biological system by detoxification. The process involves the conjugation of D-glucoronate with the toxin to make them water soluble that can be easily excreted out through urine or bile. β-Glucoronidases form a group of acid hydrolase enzymes which catalyze the hydrolysis of glucoronidase to aglycon and glucoronic acid. This process is known as deglucuronidation.

Ironically deglucuronidation due to the over expression of β-glucuronidase enzyme is associated with various pathological conditions, including tissue carcinoma, hepatic disorders, renal diseases, urinary tract infection, etc. β-Glucuronidase is reported to be released into the synovial fluid in inflammatory joint diseases, such as rheumatoid arthritis and AIDS. β-Glucuronidase is also found to be involved in the etiology of colon cancer while higher intestinal level of the enzyme is also associated with higher incidence of colon carcinoma.

### BRIEF SUMMARY OF THE INVENTION

The productionof toxic and carcinogenic metabolites may cause the tumor formation. The expression and increasedactivity of β-glucuronidase has been reported in several diseases in human, such as cancer, rheumatoid arthritis and AIDS. β-glucuronidase inhibitorsare also reported to have efficacyto decrease the onset of colonic tumors.

β-Glucuronidase (EC 3.2.1.31) is a lysosomal enzyme that cleaves β-glucuronic acid linkages from the non-reducing termini of glycosaminoglycans, such as chondroitin sulfate, heparan sulfate, and hyaluronic acid.

Lucuronidation is a defensive mechanism of the body to get rid of the poisonous chemicals by means of making them water soluble. The studies has suggested that the process of deglucuronidation is stimulated due to the hydrolysis, catalyzed by β-glucuronidase and contributes in the onset of various pathological conditions, such as tumor or carcinogenesis as well as the re-absorption of toxic chemicals. Endogenous biliary β-glucuronidasedeconjugates the glucuronides of bilirubin and causes the expansion of cholelithiasis in human bile.

Therefore, it is important to reduce the increased activity of β-glucuronidase enzyme present in various organs, body fluids, blood cells, liver, muscle, bile, spleen, kidney, gastric juice, lung, urine, and serum [1].

Plant-based β-glucuronidase inhibitors, such as 8-hydroxytricetin-7-glucuronide and isovitexin, trihydroxypipecolic acid, and scoparic acids A and C are already in clinical use [4].

The mechanisms of regulation of enzyme activity and protein targeting of β-glucuronidase have implications in the development of a variety of therapeutics [5].

Quinazolines and quinazolinone derivatives have diverse applications as chemotherapeutic agents. Several quinazolinone derivatives exhibit a multitude of interesting pharmacological activities including anticonvulsant, antidiabetic [6], analgesic [6], sedative [6] and anti-inflammatory properties [7]. Some quinazoline derivatives are also used as medications for patients with acquired cancer chemotherapy and organ transplantation [8].

During the current study an in vitro assay was employed to evaluate the β-glucuronidase inhibitory potential of a broad range of chemical compounds. Quinazolines class of compounds was also evaluated by using D-saccharic acid 1,4-lactone as standard (IC₅₀=45.75±2.16 μM). As a result, a number of quinazoline derivatives (3, 5-11, 13-16, 18-23, 25) were identified as potent inhibitor of the enzyme thus have potential to be used for the treatment of associated diseases.

To the best of our knowledge quinazoline class of compounds is reported here as novel β-glucuronidase inhibitors.

### BRIEF DESCRIPTION OF THE DRAWINGS

FIG. 1 is a graph off β-Glucuronidase Inhibitory Activity of Quinazolines

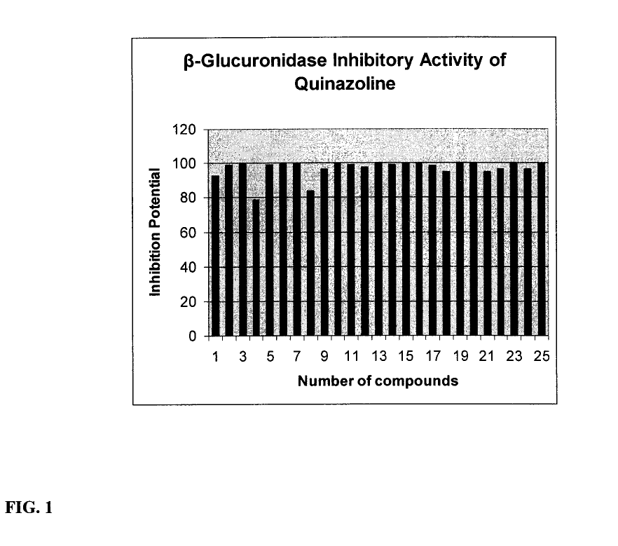

### DETAILED DESCRIPTION OF THE INVENTION

The present invention identify a novel class offi-glucuronidase inhibitors, that possesquinazoline basic skeleton, represented by formula (1)

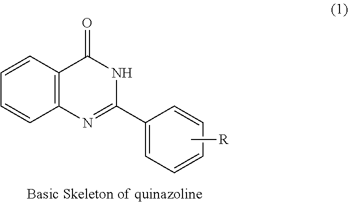

Where, R represents different substituents that caused decrease or increased in the activity Table-1).

Compounds 1-25 (IC₅₀ ranges from 0.6±0.45-198.2±2.88 μM) were evaluated for their β-glucuronidase inhibition activity and found that compounds 2 (IC₅₀=10.0±0.54 μM), 3 (IC₅₀=22.2±0.31 μM), 5 (IC₅₀=2.1±0.06 μM), 6 (IC₅₀=3.2±0.11 μM), 7 (IC₅₀=1.8±0.11 μM), 8 (IC₅₀=2.8±0.042 μM),9(IC₅₀=30.9±2.64 μM), 10 (IC₅₀=1.1±0.05 μM), 11 (IC₅₀=0.6±0.45 μM), 13 (IC₅₀=2.1±0.073 μM), 14 (IC₅₀=0.7±0.016 μM), 15 (IC₅₀=1.17±0.124 μM), 16 (IC₅₀=1.8±0.01 μM),18 (IC₅₀=37.7±1.21 μM), 19 (IC₅₀=39.8±2.88 μM), 20 (IC₅₀=1.5±0.05 μM), 22 (IC₅₀=20.1±0.92 μM),23 (IC₅₀=5.5±0.10 μM) and 25 (IC₅₀=44.0±3.12 μM) were more active than the standard, D-saccharic acid 1,4-lactone (IC₅₀=45.75±2.16 μM).

β-Glucuronidase activity was determined by the spectrophotometric method by measuring the absorbance at 405 nm ofp-nitropheno,l formed from the substrate. The total reaction volume was 250 μL. The test compound (5 μL) was dissolved in DMSO (100%), which becomes 2% in the ultimate assay (250 μL).Similar conditions were used for the standard (D-saccharic acid 1,4-lactone). The reaction mixture contained 185 μL of 0.1 M acetate buffer, 5 μL of test compound solution, 10 μL of (1U) enzyme solution was incubated at 37° C. for 30 min. The plates were read on a multiplate reader (SpectraMax plus 384, Molecular Devices, USA) at 405 nm after the addition of 50 μL of 0.4 mMp-nitrophenyl-β-D-glucuronide. All assays were run in triplicate. IC₅₀ Values were calculated by using EZ-Fit software (Perrella Scientific Inc., Amherst, Mass., USA). These values are the mean of three independent assays [1].

Cytotoxic activity of compounds (1-25) was evaluated in 96-well flat-bottomed micro-plates by using the standard MTT (3-[4,5-dimethylthiazole-2-yl]-2,5-diphenyl-tetrazolium bromide) colorimetric assay. For this purpose, PC-3 cells (Prostrate Cancer)/3T3(Mouse fibroblast) were cultured in Dulbecco's Modified Eagle's Medium, supplemented with 5% of foetal bovine serum (FBS), 100 IU/mL of penicillin and 100 μg/mL of streptomycin in 25 cm³ flask and kept in 5% CO₂ incubator at 37° C. Exponentially growing cells were harvested, counted with haemocytometer and diluted with a particular medium.

Cell culture with the concentration of 1×10⁵ cells/mL was prepared and introduced (100 μL/well) into 96-well plates. After overnight incubation, medium was removed and 200 μL of fresh medium was added with different concentrations of compounds (1-100 μM). After 72 h, 50 μL MTT (2 mg/mL) was added to each well and incubated further for 4 h. Subsequently, 100 μL of DMSO was added to each well. The extent of MTT reduction to formazan within cells was calculated by measuring the absorbance at 570 nm, using a microplate ELISA reader (Spectra Max plus, Molecular Devices, Calif., USA). The cytotoxicity was recorded as concentration causing 50% growth inhibition forPC-3/ 3T3 cells.

Synthesis of Quinazolinone derivatives (1-25)

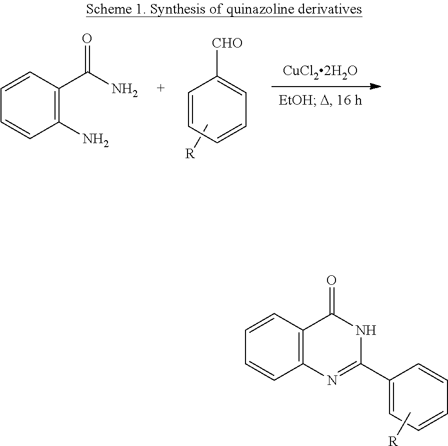

In a typical procedure,quinazolinones 1-25 were synthesized by mixing anthranilamide (2 mmol), substituted benzaldehydes (2.1 mmol) and CuCl₂.2H₂O (4 mmol) in ethanol (15 mL). The mixture was refluxed for 16 hrs., while progress of the reaction was monitored through thin layer chromatography. After completion of reaction, it was cooled to room temperature and distilled water was added until the formation of precipitates. The precipitates were filtered and washed with hexane. The yields of title compounds were found to be quantitative.

2-Phenylquinazolin-4(3H)-one (1): Yield: 0.43 g, 97%; ¹H NMR: (300 MHz, DMSO-d₆): δ_{H}12.54 (s, 1H, NH), 8.18 (m, 3H, H-5,7,8), 7.83 (d, 1H, J_{8,7}=7.2 Hz, H-8), 7.75 (d, 1H, J_{4′,3′}=J_{4′,5′}=7.8 Hz, H-4′), 7.56 (m, 4H, H-2′,6′,3′,5′); EI MS: m/z (rel. abund. %), 222 (M⁺, 83.3), 119 (100).

2-(2-Hydroxyphenyl)quinazolin-4(3H)-one(2): Yield: 0.27 g, 58%; ¹H NMR: (300 MHz, DMSO-d₆): δ_{H}13.70 (s, 1H, NH), 12.61 (s, 1H, 2′-OH), 8.23 (d, 1H, J_{5,6}=7.8 Hz, H-5), 8.16 (d, 1H, J_{6′,5′}=7.5 Hz, H-6′), 7.87 (t, 1H, J_{7(6,8)}=7.2 Hz, H-7), 7.77 (d, 1H, J_{8,7}=8.1 Hz, H-8), 7.56 (t, 1H, J_{6(5,7)}=7.2 Hz, H-6), 7.47 (t, 1H, J_{4′(3′,5′)}=7.8 Hz, H-4′), 7.01 (m, 2H, H-5′,3′);EI MS: m/z (rel. abund. %), 238 (M⁺, 100), 119 (77.5).

2-(4-Hydroxyphenyl)quinazolin-4(3H)-one (3): Yield: 0.47 g, 99%; ¹H NMR: (400 MHz, DMSO-d₆): δ_{H}12.29 (s, 1H, NH), 10.14 (s, 1H, 4′-OH), 8.11 (m, 1H, H-5), 8.03 (d, 2H, J_{2′,3′}=J_{6′,5′}=8.4 Hz, H-2′,6′), 7.80 (t, 1H, J_{7(6,8)}=7.2 Hz, H-7), 7.67 (d, 1H, J_{8,7}=8.0 Hz, H-8), 7.47 (t, 1H, J_{6(5,7)}=7.2 Hz, H-6), 6.89 (d, 2H, J_{3′,2′}=J_{5′,6′}=8.8 Hz, H-3′,5^{.});EI MS: m/z (rel. abund. %), 238 (M⁺, 100), 237 (5.4), 221 (4.6), 119 (82.6).

2-(3-Hydroxyphenyl)quinazolin-4(3H )-one (4): Yield: 0.42 g, 88%; ¹H NMR: (400 MHz, DMSO-d₆): δ_{H}12.42 (s, 1H, NH), 9.74 (s, 1H, 3′-OH), 8.14 (m, 1H, H-5), 7.84 (m, 1H, H-7), 7.72 (d, 1H, J_{8,7}=8.0 Hz, H-8), 7.59 (m, 2H, H-6,2′), 7.52 (m, 1H, H-6′), 7.34 (m, 1H, H-5′), 6.97 (m, 1H, H-4′); EI MS: m/z (rel. abund. %), 238 (M⁺, 81.6), 237 (8.5), 221 (7.3), 119 (100).

2-(3,4-Dihydroxyphenyl)quinazolin-4(3H)-one (5): Yield: 0.49 g, 96%; ¹H NMR: (400 MHz, DMSO-d₆): δ_{H}12.21 (s, 1H, NH), 9.64 (s, 1H, 4′-OH), 9.29 (s, 1H, 3′-OH), 8.10 (d, 1H, J_{5,6}=8.0 Hz, H-5), 7.78 (d, 1H, J_{8,7}=7.2 Hz, H-8), 7.66 (m, 2H, H-7,2′), 7.54 (d, 1H, J_{6′,5′}=8.0 Hz, H-6′), 7.46 (t, 1H, J_{6(5,7)}=7.6 Hz, H-6), 6.83 (d, 1H, J_{5′,6′}=8.0 Hz, H-5′); EI MS: m/z (rel. abund. %), 254 (M⁺, 8.0), 146 (100), 119 (25.5).

2-(2,5-Dihydroxyphenyl)quinazolin-4(3H)-one (6): Yield: 0.48 g, 95%; ¹1-1 NMR: (300 MHz, DMSO-d₆): δ_{H}12.60 (s, 1H, NH), 12.29 (s, 1H, 2′-OH), 9.08 (s, 1H, 5′-OH), 8.14 (d, 1H, J_{5,6}=7.5 Hz, H-5), 7.86 (t, 1H, J_{7(6,8)}=7.5 Hz, H-7), 7.73 (d, 1H, J_{8,7}=8.1 Hz, H-8), 7.60 (s, 1H, H-6′), 7.54 (t, 1H, J_{6(5,7)}=7.2 Hz, H-6), 6.92 (m, 2H, H-3′,4′); EI MS: m/z (rel. abund. %), 254 (M⁺, 100), 119 (36.3).

2-[2-Hydroxy-5-(methyloxy)phenyl]quinazolin-4(3H)-one (7): Yield: 0.44 g, 82%; ¹H NMR: (400 MHz, DMSO-d₆): δ_{H}13.42 (s, 1H, NH), 12.55 (s, 1H, 2′-OH), 8.15 (d, 1H, J_{5,6}=7.6 Hz, H-5), 7.84 (m, 3H, H-7,8,6), 7.53 (s, 1H, H-6′), 7.05 (s, 1H, H-4′), 6.94 (d, 1H, J_{3′,4′}=7.6 Hz, H-3′), 3.78 (s, 3H, 5′-OCH₃); EI MS: m/z (rel. abund. %), 268 (M⁺, 76.9), 253 (100), 119 (3.8).

2-[3-Hydroxy-4-(methyloxy)phenyl]quinazolin-4(3H)-one (8): Yield: 0.53 g, Quantitative; ¹H NMR: (400 MHz, DMSO-d₆): δ_{H}12.30 (s, 1H, NH), 9.35 (s, 1H, 3′-OH), 8.09 (s, 1H, H-5), 7.77 (m, 5H, H-7,8,6,6′,5′), 7.04 (s, 1H, H-2′), 3.85 (s, 3H, 4′-OCH₃); EI MS: m/z (rel. abund. %), 268 (M⁺, 100), 253 (25.9), 119 (82.3).

2-[2-Hydroxy-3-(methyloxy)phenyl]quinazolin-4(3H)-one (9): Yield: 0.53 g, Quantitative; ¹H NMR: (300 MHz, DMSO-d₆): δ_{H}13.94 (s, 1H, NH), 12.46 (s, 1H, 2′-OH), 8.16 (d, 1H, J_{5,6}=7.5 Hz, H-5), 7.86 (m, 3H, H-7,6,4), 7.56 (t, 1H, J_{6′(4′,5′)}=7.2 Hz, H-6′), 7.17 (d, 1H, J_{8,7}=8.1 Hz, H-8), 6.90 (t, 1H, J_{5′(4′,6′)}=8.1 Hz, H-5′), 3.82 (s, 3H, 3′-OCH₃); EI MS: m/z (rel. abund. %), 268 (M⁺, 100), 239 (50.2), 225 (53.9), 119 (39.1).

2-[4-(Methyloxy)phenyl]quinazolin-4(3H)-one (10): Yield: 0.42 g, 84%; ¹H NMR: (400 MHz, DMSO-d₆): δ_{H}12.40 (s, 1H, NH), 8.18 (m, 3H, H-5,2′,6′), 7.80 (m, 2H, H-7,8), 7.47 (s, 1H, H-6), 7.10 (d, 2H, J_{3′,2′}=J_{5′,6′}=7.6 Hz H-3′,5′), 3.85 (s, 3H, 4′-OCH₃); EI MS: m/z (rel. abund. %), 252 (M⁺, 100), 237 (3.2), 235 (2.9), 119 (64.8).

2-[3,4-Bis(methyloxy)phenyl]quinazolin-4(3H)-one (11): Yield: 0.56 g, Quantitative; ¹H NMR: (400 MHz, DMSO-d₆): δ_{H}12.43 (s, 1H, NH), 8.13 (d, 1H, J_{5,6}=7.6 Hz, H-5), 7.86 (m, 3H, H-7,8,2′), 7.71 (d, 1H, J_{8,7}=8.0 Hz, H-8), 7.49 (d, 1H, J_{6′,5′}=7.6 Hz, H-6′), 7.11 (d, 1H, J_{5′,6′}=8.4 Hz, H-5′), 3.87 (s, 3H, 4′-OCH₃), 3.83 (s, 3H, 3′-OCH₃); EI MS: m/z (rel. abund. %), 282 (M⁺, 100), 267 (21.4), 251 (25.7), 119 (20.6). 2-[3,4,5-Tris(methyloxy)phenyl]quinazolin-4(3H)-one(12) Yield: 0.61 g, 99%; ¹H NMR: (400 MHz, DMSO-d₆): δ_{H}12.51 (s, 1H, NH), 8.15 (d, 1H, f_{5,6}=7.2 Hz, H-5), 7.82 (m, 1H, H-7), 7.75 (d, 1H, J_{8,7}=8.0 Hz, H-8), 7.55 (s, 2H, H-5′,6′), 7.52 (m, 1H, H-6), 3.89 (s, 6H, 3′-OCH₃, 5′-OCH₃), 3.73 (s, 3H, 4′-OCH₃); EI MS: m/z (rel. abund. %), 312 (M⁺, 100), 297 (35.5), 281 (6.8), 119 (6.6).

2-[4-(Ethyloxy)phenyl]quinazolin-4(3H)-one (13): Yield: 0.522 g, 98%; ¹H NMR: (400 MHz, DMSO-d₆): δ_{H}12.39 (s, 1H, NH), 8.18 (d, 1H, J_{5,6}=8.7 Hz, H-5), 8.13 (d, 2H, J_{2′,3′}=J_{6′,5′}=8.1 Hz, H-2′,6′), 7.82 (t, 1H, J_{7(6,8)}=7.2 Hz, H-7), 7.70 (d, 1H, J_{8,7}=8.1 Hz, H-8), 7.49 (t, 1H, J_{6(5,7)}=7.2 Hz, H-6), 7.07 (d, 2H, J_{3′,2′}=8.7 Hz, H-3′,5′), 4.13 (q, 2H, J=14.0 Hz, 6.9 Hz, CH₂), 1.37 (t, 3H, J=6.9 Hz, CH₃); EI MS: m/z (rel. abund. %), 266 (M⁺, 100), 238 (23.1), 221 (3.7), 119 (57.2).

2-[2-(Ethyloxy)phenyl]quinazolin-4(3H)-one (14): Yield: 0.52 g, 97%; ¹H NMR: (400 MHz, DMSO-d₆): δ_{H}12.03 (s, 1H, NH), 8.14 (t, 1H, J_{5(6,7)}=6.8 Hz, H-5), 7.84 (m, 1H, H-7), 7.76 (dd, 1H, J_{6′,5′}=7.2 Hz, J_{6′,4′}=1.2 Hz, H-6′), 7.70 (d, 1H, J_{8,7}=8.0 Hz, H-8), 7.53 (m, 2H, H-6,3′), 7.18 (d, 1H, J_{3′,4′}=8.4 Hz, H-3′), 7.09 (t, 1H, J_{4′(3′.5′)}=7.2 Hz, H-4′), 4.16 (q, 2H, J=14.0 Hz, 7.2 Hz, CH₂), 1.35 (t, 3H, J=6.8 Hz, CH₃); EI MS: m/z (rel. abund. %), 266 (M⁺, 23.9), 251 (51.8), 238 (14.6), 222 (21.2), 119 (100).

2-[3-(Ethyloxy)-4-hydroxyphenyl]quinazolin-4(3H)-one (15): Yield: 0.55 g, 98%; ¹H NMR: (400 MHz, DMSO-d₆): δ_{H}12.32 (s, 1H, NH), 9.67 (s, 1H, 4′-OH), 8.11 (d, 1H, J_{5,6}=7.6 Hz, H-5), 7.77 (m, 3H, H-7,8,2), 7.68 (d, 1H, J_{6′,5′}=8.0 Hz, H-6), 7.47 (t, 1H, J_{6(5,7)}=7.2 Hz, H-6), 6.91 (d, 1H, J_{5′,6′}=8.4 Hz, H5′), 4.16 (q, 2H, J=14.0 Hz, 6.8 Hz, CH₂), 1.39 (t, 3H, J=7.2 Hz, CH₃); EI MS: m/z (rel. abund. %), 282 (M⁺, 100), 267 (29.3), 254 (80.9), 238 (11.9), 119 (46.8).

2-(2-Chlorophenyl)quinazolin-4(3H)-one (16): Yield: 0.50 g, 98%; ¹H NMR: (300 MHz, DMSO-d₆): δ_{H}12.62 (s, 1H, NH), 8.17 (d, 1H, J_{5,6}=7.2 Hz, H-5), 7.84 (s, 1H, H-6′), 7.71 (m, 6H, H-7,8,6,3′,4′,5′); EI MS: m/z (rel. abund. %), 256 (M⁺, 75.5), 239 (6.9), 221 (8.4), 119 (100).

2-(2,4-Dichlorophenyl)quinazolin-4(3H)-one (17): Yield: 0.57 g, 99%; ¹H NMR: (400 MHz, DMSO-d₆): δ_{H}12.65 (s, 1H, NH), 8.17 (d, 1H, J_{5,6}=7.6 Hz, H-5), 7.85 (m, 2H, H-7,3′), 7.71 (m, 2H, H-8,6), 7.59 (m, 2H, H-6′,5′); EI MS: m/z (rel. abund. %), 290 (M^{t}, 66.4), 273 (3.7), 255 (5.9), 220 (3.0), 119 (100).

2-(2,6-Dichlorophenyl)quinazolin-4(3H)-one (18): Yield: 0.56 g, 96%; ¹H NMR: (400 MHz, DMSO-d₆): δ_{H}12.79 (s, 1H, NH), 8.19 (d, 1H, J_{5,6}=7.2 Hz, H-5), 7.89 (m, 1H, H-7), 7.73 (d, 1H, J_{8,7}=8.0 Hz, H-8), 7.65 (m, 4H, H-6,4′,3′,5); EI MS: m/z (rel. abund. %), 290 (M⁺, 100), 273 (6.1), 255 (68.8), 220 (5.7), 119 (8.5).

2-(4-Chlorophenyl)quinazolin-4(3H)-one (19): Yield: 0.48 g, 94%; ¹H NMR: (400 MHz, DMSO-d₆): δ_{H}12.60 (s, 1H, NH), 8.20 (d, 1H, J_{5,6}=8.4 Hz, H-5), 8.15 (d, 2H, J_{3′,2′}=J_{5′,6′}=8.0 Hz, H-3′,5′), 7.86 (t, 1H, J_{7(6,8)}=7.2 Hz, H-7), 7.74 (d, 1H, J_{8,7}=8.0 Hz, H-8), 7.63 (d, 2H, J_{2′,3′}=J_{6′,5′}=8.8 Hz, H-2′,6′), 7.54 (t, 1H, J_{6(5,7)}=7.2 Hz, H-6); EI MS: m/z (rel. abund. %), 256 (M⁺, 100), 119 (89.9).

2-(2-Nitrophenyl)quinazolin-4(3H)-one (20): Yield: 0.49 g, 92%; ¹H NMR: (400 MHz, DMSO-d₆): δ_{H}12.82 (s, 1H, NH), 8.21 (m, 2H, H-3′,5), 7.90 (m, 4H, H-5′,6′,4′,7), 7.65 (d, 1H, J_{8,7}=8.4 Hz, H-8), 7.59 (t, 1H, J_{6(5,7)}=7.2 Hz, H-6); EI MS: m/z (rel. abund. %), 267 (M⁺, 100), 250 (5.5), 221 (20.8), 119 (57.6).

2-(3-Nitrophenyl)quinazolin-4(3H)-one (21): Yield: 0.34 g, 64%; ¹H NMR: (400 MHz, DMSO-d₆): δ_{H}12.87 (s, 1H, NH), 9.02 (s, 1H, H-2′), 8.61 (d, 1H, J_{4′,5′}=8.0 Hz, H-4′), 8.43 (d, 1H, J_{6′, 5}′=7.2 Hz, H-6′), 8.18 (d, 1H, J_{5′,6′}=7.6 Hz, H-5′), 7.87 (m, 3H, H-7,8,6), 7.58 (t, 1H, J_{5′(4′, 6′)}=7.6 Hz, H-5′); EI MS: m/z (rel. abund. %), 267 (M⁺, 100), 221 (78.5), 119 (29.6).

2-(4-Nitrophenyl)quinazolin-4(3H)-one (22) Yield: 0.48 g, 90%; ¹H NMR: (400 MHz, DMSO-d₆): δ_{H}12.84 (s, 1H, NH), 8.42 (m, 4H, H-3′,5′,2′,6′), 8.18 (d, 1H, J_{5,6}=8.0 Hz, H-5), 7.89 (m, 1H, H-7), 7.80 (d, 1H, J_{8,7}=7.6 Hz, H-8), 7.59 (t, 1H, J_{6(5,7)}=8 Hz, H-6); EI MS: m/z (rel. abund. %), 267 (M⁺, 100), 221 (83.1), 119 (87.3).

2-[4-(Dimethylamino)phenyl]quinazolin-4(3H)-one (23): Yield: 0.52 g, 99%; ¹H NMR: (400 MHz, DMSO-d₆): δ_{H}12.26 (s, 1H, NH), 8.10 (m, 3H, H-5,2′,6′), 7.79 (m, 1H, H-7), 7.67 (d, 1H, J_{8,7}=8.4 Hz, H-8), 7.45 (m, 1H, H-6), 6.81 (d, 2H, J_{3′,2′}=J_{5′,6′}=8.8 Hz, H-3′,5′), 3.02 (s, 6H, 4′-N(CH₃)₂); EI MS: m/z (rel. abund. %), 265 (M⁺, 100), 250 (6.1), 221 (5.2), 119 (33.5).

2-(4-Methylphenyl)quinazolin-4(3H)-one (24): Yield: 0.42 g, 89%; ¹H NMR: (400 MHz, DMSO-d₆): 802.45 (s, 1H, NH), 8.12 (m, 3H, H-5,2′,6′), 7.82 (t, 1H, J_{7(6,8)}=7.2 Hz, H-7), 7.72 (d, 1H, J_{8,7}=8.0 Hz, H-8), 7.51 (t, 1H, J_{6(5,7)}=7.6 Hz, H-6), 7.36 (d, 2H, J_{3′,2′}=J_{5′,6′}=8.4 Hz, H-3′,5′), 2.38 (s, 3H, 4LCH₃); EI MS: m/z (rel. abund. %), 236 (M⁺, 100), 219 (4.1), 119 (96.7).

2-(2-Bromo-6-hydroxyphenyl)quinazolin-4(3H)-one (25): Yield: 0.48 g, 76%; ¹H NMR: (300 MHz, DMSO-d₆): δ_{H}13.81 (s, 1H, NH), 12.57 (s, 1H, 2′-OH), 8.45 (s, 1H, H-5), 8.16 (d, 1H, J_{8,7}=6.7 Hz, H-8), 7.86 (m, 2H, H-7,6), 7.57 (m, 2H, H-4′,5′), 6.98 (d, 1H, J_{5′,4′}=6.6 Hz, H-5′); EI MS: m/z (rel. abund. %), 316 (M⁺, 100), 238 (3.7), 145 (3.8), 119 (47.2).

Anthranilamide (2 mmol), substituted benzaldehydes (2.1 mmol) and CuCl₂.2H₂O (4 mmol) in ethanol (15 mL), were added in a round bottomed flask. The mixture was refluxed for 16 h, while progress of the reaction was monitored through thin layer chromatography. After completion of reaction, it was cooled to room temperature and distilled water was added until the formation of precipitates. The precipitates were filtered and washed with hexane to afford title compounds in high yield.

We are reporting here in for the first time, some derivatives of quinazoline class of compounds, with potent β-glucuronidase inhibition activity.

We have screened twenty-five (25) compounds (1-25) against β-glucuronidase enzyme. Out of which, nineteen compounds 2-3, 5-11, 13-16, 18-20, 22, 23 and 25 showed potent activities with IC₅₀ values 10.0±0.54, 22.2±0.31, 2.1±0.06, 3.2±0.11, 1.8±0.11, 2.8±0.042, 30.9±2.64, 1.1±0.05, 0.6 ±0.45, 2.1±0.073, 0.7±0.016, 1.17±0.124, 1.8±0.01, 37.7±1.21, 39.8±2.88, 1.5±0.05, 20.1±0.92, 5.5±0.10 and 44.0±3.12 μM, respectively (Table-1). Compounds 11 (2-[3,4-bis(methyloxy)phenyl]quinazolin-4-(3H)-one) and 14 2-[2-(ethyloxy)phenyl]quinazoline-4-(3H)-one) showed excellent activities (IC₅₀=0.6±0.45 and 0.7±0.016 μM, respectively) and found more active than the standard (D-saccharic acid 1,4-lactone (IC₅₀=45.75±2.16 μM). Compounds 1, 4, 12, 17, 21 and 24 showed a weak inhibitory activity against the enzyme with IC₅₀ values between 50.3 to 172.7 μM.

Results indicate that activity of compounds may be due to the presence of different substituents on the benzene ring, attached to C-2 of the quinazoline nucleus. The discussion is given below.

o-Hydroxy substituted phenyl ring containing compound 2 showed a potent activity with IC₅₀=10.0±0.54 μM more active than the standard D-saccharicacid 1,4-lactone (IC₅₀=45.75±2.16 μM). However, compound 3 having apara-hydroxy benzene ring appeared to be less active (IC₅₀=22.2±0.31 μM) as compared to o-hydroxy benzene ring containing compound 2(IC₅₀=10.0±0.54 μM).Further decrease in activity was observed in compound 4 having meta-hydroxy benzene ring (IC₅₀ =198.2±2.88 μM). Therefore, hydroxyl group at ortho and para positions of benzene ring are found to be beneficial for inhibition offi-glucuronidase activity.

Significant increase in inhibition activities of compounds 5 and6 was observed with IC₅₀ values 2.1±0.06 and 3.2±0.11 μM respectively,when hydroxyls were present at meta, para and ortho positions adjacent to each other.

Introduction of methoxy group found to be beneficial as well andthe activities of derivatives 7-11,13, 14 and 15wit IC₅₀ values1.8±0.11, 2.8±0.042, 30.9±2.64, 1.1±0.05, 0.6±0.45, 2.1±0.073, 0.7±0.016 and 1.17±0.124 μM, respectively. All these compounds found to be more active than standard D-saccharicacidl,4-lactone (IC₅₀=45.75±2.16 μM). The comparison of the activities of methoxy substituted compounds 7, 8, 10 and 11 and ethoxy substituted compounds 14 and 15with their hydroxyl substituted analogues (2, IC₅₀=10.0±0.54 μM), (3, IC₅₀=22.2±0.31 μM), (4, IC₅₀=198.2±2.88 μM), (5, IC₅₀=2.1±0.06 μM) and (6, IC₅₀=3.2±0.11 μM) showed that alkoxy group (—OCH₃/—OC₂H₅) are responsible for more potent activities. However o,m,p-tri-methoxy substituted phenyl ring containing compound 12 was found to be least active among the series with IC₅₀=120.5±2.54

Introduction of chlorine on the phenyl ring attached at C-2 of quinazoline ring is also found to be responsible for potent activities as observed in compounds 16-19 (IC₅₀=1.8±0.017, 39.8±2.88, 37.7±1.21 and 61.03±6.26 μM, respectively). It was also observed that chlorosubstituted phenyl ring containing compound 16 (IC₅₀=1.8±0.017 μM) is found to be most active. The activities of di-orthochloro and m-chloro substituted phenyl ring containing compounds have shown almost similar inhibitory potential with IC₅₀ values39.8±2.88 and 37.7±1.21, respectively.

Compound 20 with substitution at ortho position of phenyl ring showed highly potent activity with IC₅₀=1.5±0.05 pM, while in compound 22 having p-nitro phenyl ring showed less inhibitory potential with IC₅₀=20.1±0.92 μM. Further decrease in inhibitory potential was observed in compound 21 having am-nitro phenyl ring (IC₅₀=50.4±1.40 μM).

TABLE-1
β-Glucuronidase Inhibitory Activity of Quinazolines

| | % | IC₅₀ |
|---|---|---|
| Structures | Inhibition | (μM) ± SEM |
| 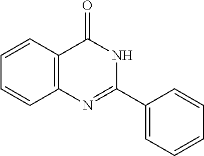 [SMILES: O=c1[nH]c(-c2ccccc2)nc2ccccc12] | 92.5 | 177.0 ± 5.04 |
| 2-Phenylquinazoline-4(3H)-one (1) | | |
| 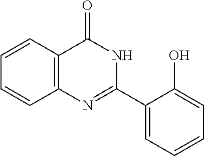 [SMILES: O=c1[nH]c(-c2ccccc2O)nc2ccccc12] | 99.1 | 10.0 ± 0.54* |
| 2-(2-Hydroxy-phenylquinazoline-4(3H)- | | |
| one (2) | | |
| 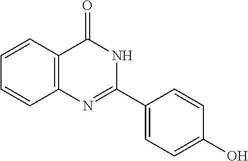 [SMILES: O=c1[nH]c(-c2ccc(O)cc2)nc2ccccc12] | 99.6 | 22.2 ± 0.31* |
| 2-(4-Hydroxy phenyl)quinazoline- | | |
| 4(3H)-one (3) | | |
| 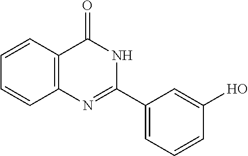 [SMILES: O=c1[nH]c(-c2cccc(O)c2)nc2ccccc12] | 78.5 | 198.2 ± 2.88 |
| 2-(3-Hydroxyphenyl)quinazoline-4(3H)- | | |
| one (4) | | |
| 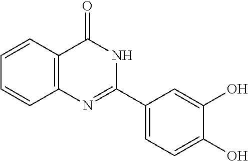 [SMILES: O=c1[nH]c(-c2ccc(O)c(O)c2)nc2ccccc12] | 98.8 | 2.1 ± 0.06* |
| 2-(3,4-Dihydroxyphenyl)quinazoline- | | |
| 4(3H)-one (5) | | |
| 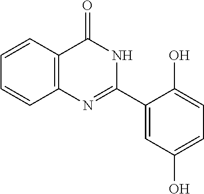 [SMILES: O=c1[nH]c(-c2cc(O)ccc2O)nc2ccccc12] | 99.9 | 3.2 ± 0.11* |
| 2-(2,5-Dihydroxyphenyl)-quinazoline- | | |
| 4(3H)-one (6) | | |
| 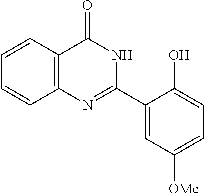 [SMILES: COc1ccc(O)c(-c2nc3ccccc3c(=O)[nH]2)c1] | 99.4 | 1.8 ± 0.11* |
| 2-[2-Hydroxy-5-(methyloxyphenyl]- | | |
| quinazoline-4(3H)-one (7) | | |
| 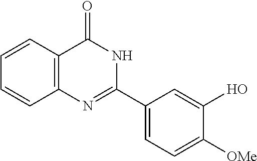 [SMILES: COc1ccc(-c2nc3ccccc3c(=O)[nH]2)cc1O] | 84.0 | 2.8 ± 0.042* |
| 2-[3-Hydroxyl-4- | | |
| (methyloxy)phenyl]quinazoline-4(3H)- | | |
| one (8) | | |
| 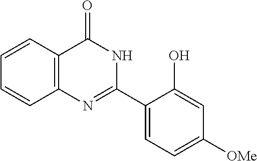 [SMILES: COc1ccc(-c2nc3ccccc3c(=O)[nH]2)c(O)c1] | 96.5 | 30.9 ± 2.64* |
| 2-[2-Hydroxy-3- | | |
| (methyloxy)phenyl]quinazoline-4(3H)- | | |
| one (9) | | |
| 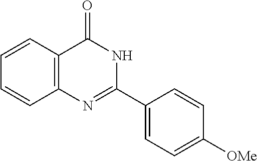 [SMILES: COc1ccc(-c2nc3ccccc3c(=O)[nH]2)cc1] | 99.7 | 1.1 ± 0.05* |
| 2-(4-Methoxy phenyl)quinazoline-4- | | |
| (3H)-one (10) | | |
| 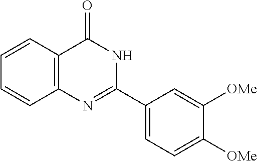 [SMILES: COc1ccc(-c2nc3ccccc3c(=O)[nH]2)cc1OC] | 98.8 | 0.6 ± 0.45* |
| 2-[3,4-Bis(methyloxy)phenyl]quinazolin- | | |
| 4(3H)-one (11) | | |
| 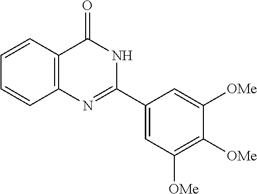 [SMILES: COc1cc(-c2nc3ccccc3c(=O)[nH]2)cc(OC)c1OC] | 97.6 | 120.5 ± 2.54 |
| 2-[3,4,5-Tris | | |
| (methyloxy)phenyl]quinazolin-4(3H)-one | | |
| (12) | | |
| 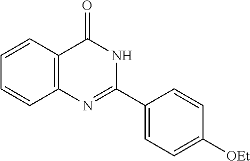 [SMILES: CCOc1ccc(-c2nc3ccccc3c(=O)[nH]2)cc1] | 99.9 | 2.1 ± 0.073* |
| 2-[4-(Ethyloxy)phenyl] quinazolin-4(3H)- | | |
| one (13) | | |
| 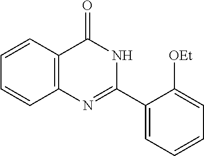 [SMILES: CCOc1ccccc1-c1nc2ccccc2c(=O)[nH]1] | 98.9 | 0.7 ± 0.016* |
| 2-[2-(Ethyloxy)phenyl]quinazoline- | | |
| 4(3H)-one (14) | | |
| 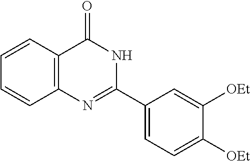 [SMILES: CCOc1ccc(-c2nc3ccccc3c(=O)[nH]2)cc1OCC] | 99.8 | 1.17 ± 0.124* |
| 2-[3-(Ethyloxy)-4-hydroxy | | |
| phenyl]quinazoline-4(3H)-one (15) | | |
| 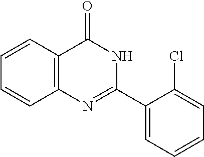 [SMILES: O=c1[nH]c(-c2ccccc2Cl)nc2ccccc12] | 99.6 | 1.8 ± 0.017* |
| 2-(2-Cholrophenyl)quinazoline-4(3H)- | | |
| one (16) | | |
| 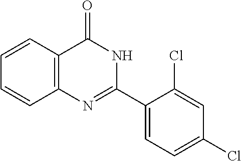 [SMILES: O=c1[nH]c(-c2ccc(Cl)cc2Cl)nc2ccccc12] | 98.4 | 61.03 ± 6.26 |
| 2-(2,4-Dicholrophenyl)quinazoline- | | |
| 4(3H)-one (17) | | |
| 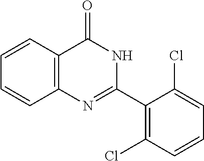 [SMILES: O=c1[nH]c(-c2c(Cl)cccc2Cl)nc2ccccc12] | 94.6 | 37.7 ± 1.21* |
| 2-(2,6-Dicholro phenyl)quinazolin-4(3H)- | | |
| one (18) | | |
| 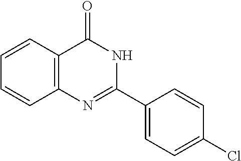 [SMILES: O=c1[nH]c(-c2ccc(Cl)cc2)nc2ccccc12] | 99.8 | 39.8 ± 2.88* |
| 2-(4-Cholro phenyl)quinazoline-4(3H)- | | |
| one (19) | | |
| 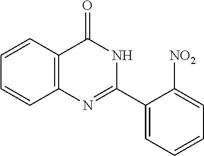 [SMILES: O=c1[nH]c(-c2ccccc2[N+](=O)[O-])nc2ccccc12] | 99.6 | 1.5 ± 0.05* |
| 2-(2-Nitrophenyl)quinazoline-4(3H)-one | | |
| (20) | | |
| 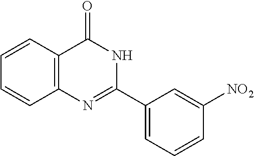 [SMILES: O=c1[nH]c(-c2cccc([N+](=O)[O-])c2)nc2ccccc12] | 95.1 | 50.4 ± 1.40 |
| 2-(3-Nitro phenyl)quinazoline-4(3H)-one | | |
| (21) | | |
| 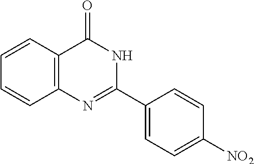 [SMILES: O=c1[nH]c(-c2ccc([N+](=O)[O-])cc2)nc2ccccc12] | 96.4 | 20.1 ± 0.92* |
| 2-(4-Nitrophenyl)quinazoline-4(3H)-one | | |
| (22) | | |
| 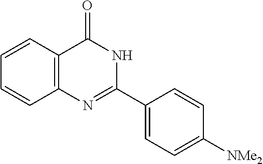 [SMILES: CN(C)c1ccc(-c2nc3ccccc3c(=O)[nH]2)cc1] | 99.6 | 5.5 ± 0.10* |
| 2-[4-(Dimethyl amine)phenyl] | | |
| quinazolin-4(3H)-one (23) | | |
| 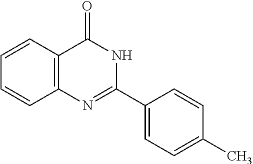 [SMILES: Cc1ccc(-c2nc3ccccc3c(=O)[nH]2)cc1] | 96.3 | 172.7 ± 4.84 |
| 2-(4-Methyl phenyl)quinazoline-4(3H)- | | |
| one (24) | | |
| 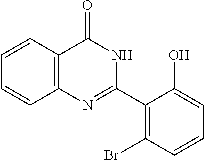 [SMILES: O=c1[nH]c(-c2c(O)cccc2Br)nc2ccccc12] | 99.7 | 44.0 ± 3.12* |
| 2-(2-Bromo-6-hydroxy | | |
| phenyl)quinazoline-4(3H)-one (25) | | |

*Values lower than standard (i.e. D-saccharic acid 1,4-lactone, IC₅₀ = 45.75 ± 2.16 μM)

Compounds 1-25 were tested against PC-3 cells for cytotoxicity the results are shown in Table-2. The IC₅₀ values of compounds showed no cytotoxicity effects towards PC-3 cells.

TABLE 2
Cytotoxicity of Compounds 1-25 for Cytotoxicity
Against PC-3 Cells In Vitro

| Compounds | IC₅₀ (μM) ± SEM |
|---|---|
| 1 | >30 |
| 2 | >30 |
| 3 | >30 |
| 4 | >30 |
| 5 | >30 |
| 6 | >30 |
| 7 | >30 |
| 8 | >30 |
| 9 | >30 |
| 10 | >30 |
| 11 | >30 |
| 12 | >30 |
| 13 | >30 |
| 14 | >30 |
| 15 | >30 |
| 16 | >30 |
| 17 | >30 |
| 18 | >30 |
| 19 | >30 |
| 20 | >30 |
| 21 | >30 |
| 22 | >30 |
| 23 | >30 |
| 24 | >30 |
| 25 | >30 |

Standard (Doxorubicin, IC₅₀ = 0.912 ± 0.12 μM)

1. A method of inhibiting β-Glucuronidase enzyme by contacting humans or animals with a suitable quantity of (2-[3,4-bis(methyloxy)phenyl]quinazolin-4-(3H)-one) or (2-[2-(ethyloxy)phenyl]quinazoline-4-(3H)-one) or a combination thereof.

2. (canceled)
